(19) United States
(12) Patent Application Publication
(10) Pub. No.: US 2026/0241976 A1
(43) Pub. Date: Aug. 20, 2026

(54) ELECTRODE DEVICE AND STEERING WHEEL

(71) Applicant: Panasonic Automotive Systems Co., Ltd., Kanagawa (JP)

(72) Inventors: Yasuhiro YAMASHITA, Osaka (JP); Hideaki ETO, Osaka (JP)

(73) Assignee: Panasonic Automotive Systems Co., Ltd., Kanagawa (JP)

(21) Appl. No.: 19/438,862

(22) Filed: Jan. 2, 2026

(30) Foreign Application Priority Data
Feb. 14, 2025 (JP) 2025-022079

(51) Int. Cl. B62D 1/04; B62D 1/06

(52) U.S. Cl. CPC B62D 1/046; B62D 1/06

(57) ABSTRACT

An electrode device is to be disposed partially exposed from a surface of a steering wheel provided in a vehicle, and the electrode device includes an electrode, a base on which the electrode is disposed, and an exterior member disposed on the base to support the electrode. The base or the exterior member includes an engaging portion that engages with the steering wheel, the electrode is exposed from the exterior member such that the exterior member forms a portion of an outer peripheral surface of the steering wheel when the electrode device is disposed in the steering wheel.

### CROSS REFERENCE TO RELATED APPLICATION

The present application is based on and claims priority of Japanese Patent Application No. 2025-022079 filed on February 14, 2025.

### FIELD

The present disclosure relates to an electrode device and a steering wheel.

### BACKGROUND

As a related technique, there is known a conductive rubber-coated fabric that measures a heartbeat signal of a driving driver from an electrode provided in a vehicle’s steering wheel.

For example, Patent Literature (PTL) 1 discloses a conductive rubber-coated fabric that can be used as a biosensing electrode. The conductive rubber-coated fabric includes a conductive base fabric layer formed by a conductive base fabric and having a first conductive base fabric layer surface, and a conductive rubber layer formed by laminating conductive rubber on the first conductive base fabric layer surface.

### Citation List

### Patent Literature

PTL 1: International Patent Publication No. 2022-075129

### SUMMARY

However, the conductive rubber-coated fabric according to PTL 1 can be improved upon.

In view of this, the present disclosure provides an electrode device capable of improving upon the above related art.

An electrode device according to one aspect of the present disclosure is an electrode device to be disposed partially exposed from a surface of a steering wheel provided in a vehicle, and the electrode device includes: an electrode; a base on which the electrode is disposed; and an exterior member disposed on the base to support the electrode, wherein the base or the exterior member includes an engaging portion that engages with the steering wheel, the exterior member forms a portion of an outer peripheral surface of the steering wheel, when the electrode device is disposed in the steering wheel, and the electrode is exposed from the exterior member to form a portion of the outer peripheral surface.

The electrode device and so forth according to the present disclosure are capable of improving upon the above related art.

### BRIEF DESCRIPTION OF DRAWINGS

These and other advantages and features of the present disclosure will become apparent from the following description thereof taken in conjunction with the accompanying drawings that illustrate a specific embodiment of the present disclosure.

FIG. 1 is a schematic diagram showing one example of a vehicle in which a steering wheel according to an embodiment is provided.

FIG. 2 is a sectional view showing the steering wheel, taken along the A-A line indicated in FIG. 1.

FIG. 3 is a sectional view showing a manner in which an electrode device is mounted in a recess formed in the steering wheel according to the embodiment.

FIG. 4 is an exploded sectional view showing the electrode device.

FIG. 5 is a plan view showing a front surface of the electrode device.

FIG. 6 is a plan view showing a back surface of the electrode device.

FIG. 7 is a sectional view showing a steering wheel provided with an electrode device laminated with an outer cover material.

FIG. 8 is a sectional view showing an electrode device in which an electrode and a wire are directly connected to each other.

FIG. 9 is a plan view showing a back surface of the electrode device shown in FIG. 8.

FIG. 10 is a sectional view showing a steering wheel provided with an electrode device in which an engaging portion engages with a metal core.

FIG. 11 is a plan view showing a back surface of the electrode device shown in FIG. 10.

FIG. 12 is a sectional view showing another steering wheel provided with an electrode device in which an engaging portion engages with a metal core.

FIG. 13 is a sectional view showing yet another steering wheel provided with an electrode device in which an engaging portion engages with a metal core.

FIG. 14 is a sectional view showing a steering wheel provided with an electrode device that is disposed on the side that faces toward the front of the vehicle.

FIG. 15 is a sectional view showing a steering wheel provided with an electrode device that is disposed on the side that faces toward the rear of the vehicle.

FIG. 16 is a diagram showing a range of an electrode disposed on a rim in its neutral position.

### DESCRIPTION OF EMBODIMENT

Hereinafter, an embodiment will be described in concrete terms with reference to the drawings.

It is to be noted that the embodiment described below merely illustrates general or specific examples. The numerical values, the shapes, the materials, the constituent elements, the arrangement positions and the connection modes of the constituent elements, and so on illustrated according to the following embodiment are examples and are not intended to limit the present disclosure. In addition, among the constituent elements described according to the following embodiment, any constituent elements that are not cited in the independent claims are to be construed as optional constituent elements.

Furthermore, the drawings are schematic diagrams and do not necessarily provide exact depictions. In addition, in the drawings, any constituent elements that are identical are given identical reference characters.

In the following description of the embodiment, expressions such as “T shape” or “T-shaped” are used. For example, the expression “T shape” means not only that an element is shaped exactly like the letter T but also that an element is shaped substantially like the letter T. In other words, this expression allows for an error of several percentages. Furthermore, the expression “T shape” means that an element is T-shaped within the range in which the advantageous effects of the present disclosure can be achieved. The explanation above applies in a similar manner to other expressions with the term “shape” or “shaped”.

### Embodiment

### Configuration

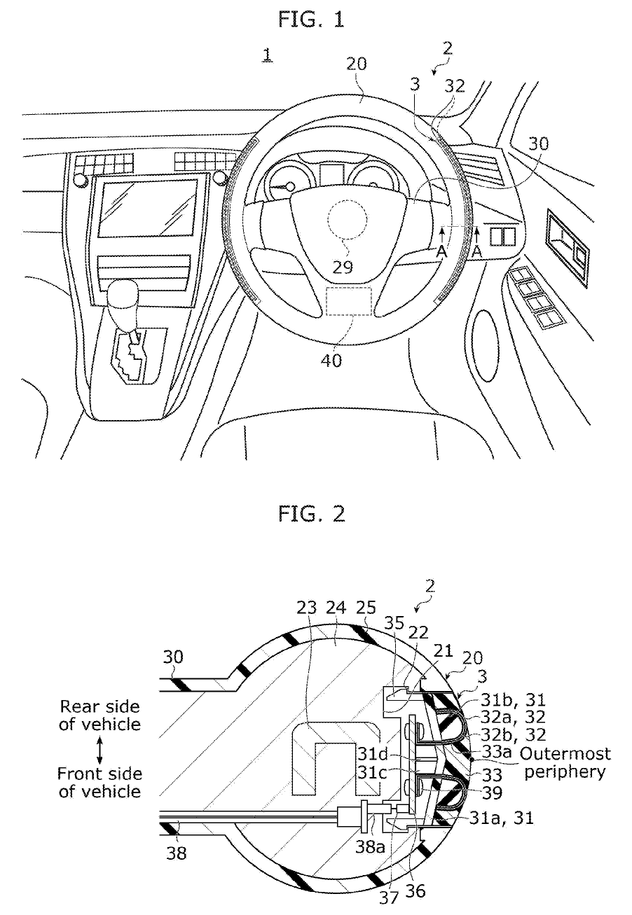

First, steering wheel 2 provided in vehicle 1 will be described with reference to FIG. 1 to FIG. 6.

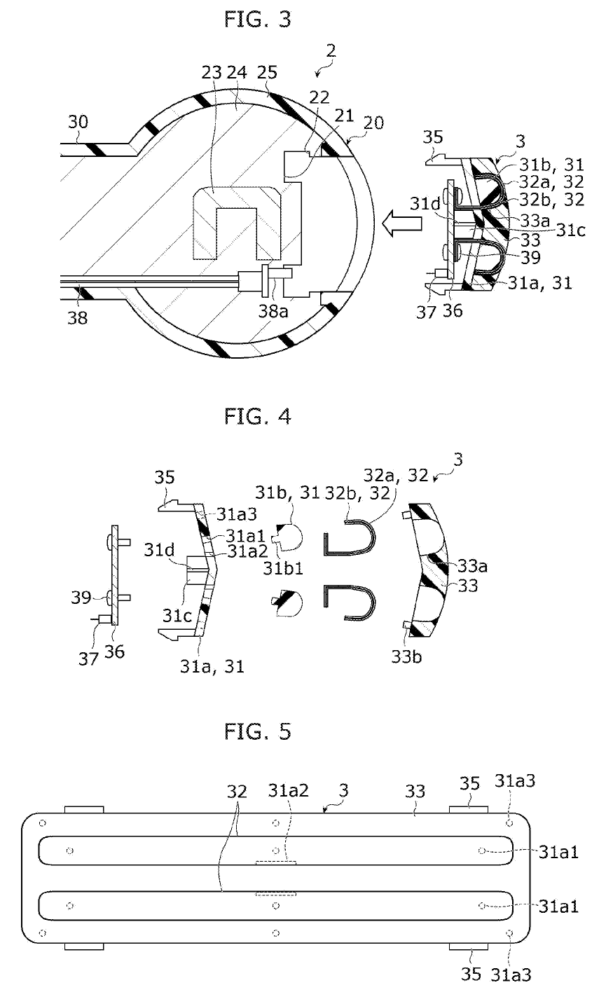

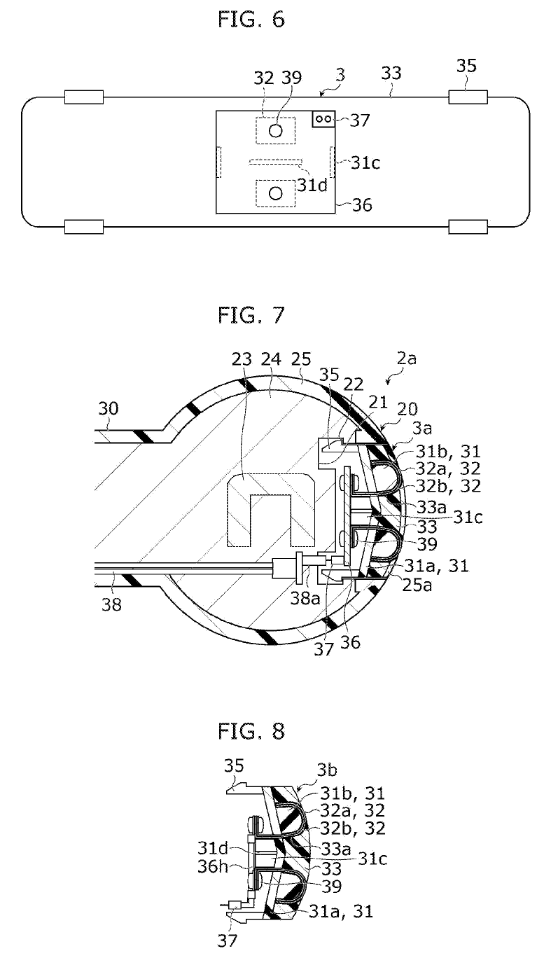

FIG. 1 is a schematic diagram showing one example of vehicle 1 in which steering wheel 2 according to an embodiment is provided. FIG. 2 is a sectional view showing steering wheel 2, taken along the A-A line indicated in FIG. 1. FIG. 3 is a sectional view showing a manner in which electrode device 3 is mounted in recess 21 of steering wheel 2 according to the embodiment. FIG. 4 is an exploded sectional view showing electrode device 3. FIG. 4 shows a state held before connector 39 electrically connects electrode 32 to circuit board 36. FIG. 5 is a plan view showing a front surface of electrode device 3. FIG. 6 is a plan view showing a back surface of electrode device 3.

As shown in FIG. 1 and FIG. 2, steering wheel 2 can provide a steering angle to the steered wheels of vehicle 1. Steering wheel 2 includes rim 20, spoke 30, hub 29, electrode device 3, and controller 40.

Rim 20 is a gripping part that the driver (a person) grips with his or her hands. Rim 20 shown in FIG. 1 has, for example, a ring shape. It is to be noted that the shape of rim 20 is not limited to a ring shape, and rim 20 may have any shape, such as a yoke shape, a D shape, or an elliptical shape.

As shown in FIG. 2, rim 20 includes metal core 23, foam 24 covering metal core 23, and outer cover material 25 covering foam 24.

Foam 24 constitutes the outer shell of rim 20 of steering wheel 2. Foam 24 is made of a urethane resin material, such as polyurethane. Foam 24 embeds metal core 23 therein.

Outer cover material 25 has an elongated sheet form that extends in the circumferential direction of rim 20 and covers foam 24. Steering wheel 2 has recess 21 formed therein, and thus outer cover material 25 covers foam 24 except at recess 21.

As shown in FIG. 1, spoke 30 is a support member that connects rim 20 and hub 29. Spoke 30 is formed by metal core 23 and foam 24 that covers metal core 23. Spoke 30 is disposed on the inner circumferential surface of rim 20. Spoke 30, connected to rim 20, forms a T shape along with rim 20.

Specifically, as shown in FIG. 1 and FIG. 3, when rim 20 in its neutral position is viewed as a clock, spoke 30 on the right side extends in the 3-o’clock direction from hub 29, spoke 30 on the left side extends in the 9-o’clock direction from hub 29, and spoke 30 on the lower side extends in the 6-o’clock direction from hub 29.

Hub 29 is a member that transmits the rotary force produced as rim 20 is operated to the steering shaft and is located in the center portion of steering wheel 2.

As shown in FIG. 2 and FIG. 3, electrode device 3 is disposed in recess 21 formed in the surface of steering wheel 2 of vehicle 1 in such a manner that a portion of electrode device 3 is exposed from the surface of steering wheel 2. In other words, electrode device 3 is mounted in recess 21 so as to fill recess 21 formed in rim 20. Electrode device 3 can be attached to or detached from rim 20.

Recess 21 is an elongated groove formed along the circumferential direction of rim 20. Since recess 21 is an elongated groove, electrode device 3 also has an elongated shape that corresponds to the shape of recess 21.

When rim 20 in its neutral position is viewed from the front, recess 21 is formed in each of the right side and the left side of rim 20. When rim 20 in its neutral position is viewed as a clock, one recess 21 is formed in the area of rim 20 that corresponds to the range of from around 2 o’clock to around 4 o’clock, and another recess 21 is formed in the area of rim 20 that corresponds to the range of from around 8 o’clock to around 10 o’clock. These areas correspond to the positions where the driver may grip rim 20 with his or her hands. In this example, the right side and the left side of rim 20 as rim 20 in its neutral position is viewed from the front may also be referred to simply as the right side and the left side of rim 20.

Recess 21 is formed in the outermost periphery of steering wheel 2. Therefore, electrode device 3 mounted in recess 21 becomes located in the outermost periphery of steering wheel 2.

Electrode device 3 is capable of detecting biometric information of the driver touching steering wheel 2 when the driver grips steering wheel 2 with his or her hands. The driver’s biometric information includes information indicating the heart rate of the driver touching steering wheel 2, information indicating the perspiration state of the driver touching steering wheel 2, and information indicating whether the driver is touching steering wheel 2.

Next, a specific configuration of electrode device 3 will be described.

Electrode device 3 includes base 31, electrode 32, and cover 33.

Base 31 is a support member for disposing electrode 32 thereon. Base 31 is elongated in the circumferential direction of rim 20.

Base 31 includes base body 31a and electrode supporter 31b.

Base body 31a is a support member for disposing cover 33 and electrode supporter 31b thereon. Base body 31a is elongated in the circumferential direction of rim 20. When electrode device 3 is viewed in section as electrode device 3 is cut along a plane orthogonal to the circumferential direction of rim 20, base body 31a according to the present embodiment has a V shape that is bent at the center portion in the widthwise direction or a curved U shape.

Electrode supporter 31b is a support member for disposing electrode 32 thereon. Electrode supporter 31b is elongated in the lengthwise direction of base body 31a and is a protruding member disposed so as to protrude from the front surface of base body 31a. According to the present embodiment, when electrode device 3 is viewed in section, electrode supporter 31b is a protruding member having a semicircular protrusion.

Specifically, as shown in FIG. 4, base body 31a has formed therein first engaging hole 31a1 that engages with electrode supporter 31b. Electrode supporter 31b has first engaging protrusion 31b1 that engages with first engaging hole 31a1. Therefore, as first engaging hole 31a1 and first engaging protrusion 31b1 engage with each other, electrode supporter 31b becomes disposed on the front surface of base body 31a as a protruding member. According to the present embodiment, a pair of electrode supporters 31b is disposed on the front surface of base body 31a.

Cover 33 is an exterior member for covering base 31. Cover 33 is formed, for example, of a resin material and may be formed of a material different from outer cover material 25. Cover 33 is elongated in the lengthwise direction of base body 31a.

Cover 33 is disposed on base 31 so as to support electrode 32. Specifically, cover 33 is laminated on the front surface of base body 31a and disposed on the front surface of base body 31a. Base body 31a has further formed therein second engaging hole 31a3 that engages with cover 33. Cover 33 includes second engaging protrusion 33b that engages with second engaging hole 31a3. Therefore, as second engaging hole 31a3 and second engaging protrusion 33b engage with each other, electrode supporter 31b becomes disposed on the front surface of base body 31a as a protruding member. Cover 33 may be one example of an exterior member.

When electrode device 3 is disposed in recess 21, cover 33 forms a portion of the outer peripheral surface of steering wheel 2. In other words, cover 33 is disposed such that the outer peripheral surface of steering wheel 2 is flush with the surface of cover 33 of electrode device 3. Therefore, when the driver grips steering wheel 2, the driver is less likely to have a sense of discomfort caused by the border between electrode device 3 and rim 20 of steering wheel 2.

Cover 33 has formed therein slit 33a for exposing electrode 32 therethrough. Slit 33a is formed in cover 33 so as to correspond in position and in size to electrode 32 and electrode supporter 31b. One slit 33a is formed in cover 33 in the area on rim 20 that corresponds to the range of from around 2 o’clock to around 4 o’clock, and another slit 33a is formed in cover 33 in the area on rim 20 that corresponds to the range of from around 8 o’clock to around 10 o’clock.

Electrode 32 and electrode supporter 31b are disposed inside slit 33a. In other words, electrode 32 is disposed in slit 33a in a state in which electrode 32 is sandwiched between cover 33 and electrode supporter 31b. This configuration makes it possible for slit 33a to support electrode 32 such that electrode 32 is not separated from electrode supporter 31b.

In particular, slit 33a is formed so as to correspond in size and in shape to electrode 32 and electrode supporter 31b. Therefore, when electrode device 3 is viewed in section, slit 33a is formed so as to fit in the direction normal to the surface of base body 31a. Accordingly, this configuration makes it possible for cover 33 to support electrode 32 such that electrode 32 is not separated from electrode supporter 31b.

According to the present embodiment, a pair of electrodes 32 and a pair of electrode supporters 31b are disposed on base body 31a. Therefore, cover 33 has formed therein a pair of slits 33a that corresponds, one to one, to the pair of electrodes 32 and also corresponds, one to one, to the pair of electrode supporters 31b.

Electrode 32 is a sensor electrode for detecting biometric information of the driver touching steering wheel 2 when the driver grips steering wheel 2 with his or her hand. Electrode 32 is elongated in the lengthwise direction of electrode supporter 31b.

Electrode 32 is laminated on electrode supporter 31b of base 31 and disposed on the surface of electrode supporter 31b. Electrode 32 is exposed from cover 33 so as to form a portion of the outer peripheral surface of rim 20 of steering wheel 2. In other words, a portion of electrode 32 is exposed through slit 33a formed in cover 33. It is to be noted that, according to the present embodiment, electrode 32 is exposed from cover 33 so as to be flush with the outer peripheral surface of rim 20 of steering wheel 2. In other words, electrode 32 is exposed through slit 33a formed in cover 33. It is to be noted that, for example, according to the present embodiment, a step of less than 1 mm is considered to be flush.

Electrode 32 is electrically connected, via base body 31a, to circuit board 36 that is disposed on the back surface of base body 31a. According to the present embodiment, electrode 32 is electrically connected to circuit board 36 by connector 39 formed, for example, of a rivet or a conductive adhesive.

Specifically, as shown in FIG. 4 and FIG. 5, base body 31a has formed therein insertion hole 31a2 into which electrode 32 is to be inserted. A portion of electrode 32 extends from the surface of electrode supporter 31b to the back surface of base body 31a via insertion hole 31a2 and is electrically connected to circuit board 36 that is disposed on the back surface of base body 31a.

As shown in FIG. 1 and FIG. 2, circuit board 36 is electrically connected to wire 38 that is disposed inside steering wheel 2, and thus electrode 32 is electrically connected to wire 38 via circuit board 36. Wire 38 electrically connects circuit board 36 and controller 40.

As shown in FIG. 3 and FIG. 4, since electrode device 3 includes the pair of electrodes 32, partition plate 31d is formed between the pair of electrodes 32 on the back surface of base body 31a so that the pair of electrodes 32 that has reached the back surface of base body 31a does not come into contact with each other.

Electrode 32 includes conductive layer 32a formed of a conductive elastomer or a metal plate. Electrode 32 may further include conductive base fabric 32b. In this case, when conductive layer 32a and conductive base fabric 32b are disposed on the surface of electrode supporter 31b, conductive base fabric 32b is laminated on the surface of electrode supporter 31b, and conductive layer 32a is disposed on the surface of conductive base fabric 32b that is opposite to the side that faces electrode supporter 31b. Therefore, conductive layer 32a is disposed such that a portion thereof is exposed through slit 33a in cover 33. In the example described according to the present embodiment, electrode 32 includes conductive layer 32a and conductive base fabric 32b.

According to the present embodiment, the pair of electrodes 32 is disposed on the surfaces of the pair of electrode supporters 31b such that the pair of electrodes 32 corresponds, one to one, to the pair of electrode supporters 31b.

The pair of electrodes 32 extends in the direction in which electrode supporters 31b extend and is disposed adjacent to each other such that the pair of electrodes 32 does not come into contact with each other. In this example, that the pair of electrodes 32 is disposed adjacent to each other means, for example, that the space between the pair of electrodes 32 is narrower than the thickness of the driver’s fingers so that the fingers can make contact across the pair of electrodes 32 when his or her hand comes into contact with steering wheel 2.

According to the present embodiment, since electrode device 3 is located at the outermost periphery of rim 20, as shown in FIG. 2, the pair of electrodes 32 is disposed on rim 20 such that the outermost periphery of rim 20 passes between the pair of electrodes 32. One electrode 32 of the pair of electrodes 32 is disposed on the side that faces toward the front of vehicle 1, and other electrode 32 of the pair of electrodes 32 is disposed on the side that faces toward the rear of vehicle 1.

Base 31 or cover 33 includes engaging portion 35 that engages with steering wheel 2. In an example according to the present embodiment, engaging portion 35 is formed on base 31. Engaging portion 35 extends in a direction that intersects with the circumferential direction of rim 20 and so as to approach hub 29 of steering wheel 2. Engaging portion 35 is a hook-shaped hook member that engages, for example, with engagement receiving portion 22 formed in recess 21.

Furthermore, as shown in FIG. 2 and FIG. 6, electrode device 3 may further include circuit board 36.

Circuit board 36 may be disposed so as to overlap base body 31a. Circuit board 36 may be supported by support portion 31c disposed on the back surface of base body 31a. Support portion 31c may protrude from the back surface of base body 31a toward metal core 23.

The pair of electrodes 32 may be disposed on the front surface of circuit board 36, and circuit board 36 may be electrically connected to the pair of electrodes 32 via connector 39. Terminal 37 may be disposed on the back surface of circuit board 36, and terminal 37 may be electrically connected to terminal 38a of the pair of wires 38 disposed inside steering wheel 2. The pair of wires 38 is bundled together, and terminal 38a is provided at the end of that bundle. One wire 38 of the pair of wires 38 is electrically connected to one of the pair of electrodes 32 via terminal 38a and terminal 37, and other wire 38 of the pair of wires 38 is electrically connected to the other of the pair of electrodes 32 via terminal 38a and terminal 37.

Furthermore, as shown in FIG. 1, steering wheel 2 may include controller 40. Controller 40 may detect, for example but not limited to, the driver’s heart rate and perspiration state based on the biometric information detected by the pair of electrodes 32.

For example, one of the pair of electrodes 32 that is disposed on the right side of rim 20 or the left side of rim 20 may serve as a heartbeat detecting electrode, and the other of the pair of electrodes 32 that is disposed on the right side of rim 20 or the left side of rim 20 may serve as a reference electrode. Furthermore, one of the pair of electrodes 32 that is disposed on the left side of rim 20 or the right side of rim 20 may serve as a heartbeat and perspiration detecting electrode, and the other of the pair of electrodes 32 that is disposed on the left side of rim 20 or the right side of rim 20 may serve as a perspiration detecting electrode.

In this case, controller 40 may include a heartbeat detecting circuit, a perspiration detecting circuit, and a grip detecting circuit.

The heartbeat detecting circuit is a heart rate monitor that, upon the driver’s hand making contact with steering wheel 2, detects the driver’s heart rate. The heartbeat detecting circuit detects the driver’s heart rate based on signals output from the heartbeat detecting electrode, the heartbeat and perspiration detecting electrode, and the reference electrode, and can thus obtain information indicating the driver’s heart rate. The heartbeat detecting circuit may display information indicating the driver’s heart rate on an in-vehicle monitor provided in vehicle 1.

The perspiration detecting circuit is a perspiration detector that, upon the driver’s hand making contact with steering wheel 2, can detect the perspiration state of the driver’s hand. The perspiration detecting circuit can detect the perspiration state of the driver’s hand as perspiration signals output from the perspiration detecting electrode and the heartbeat and perspiration detecting electrode are input to the perspiration detecting circuit. The perspiration detecting circuit can display information indicating the driver’s perspiration state on an in-vehicle monitor provided in vehicle 1.

With controller 40 configured as described above, the driver’s emotion can be estimated based on the driver’s heart rate and perspiration state. Based on the driver’s emotion estimated by controller 40, an electronic control unit (ECU) provided in vehicle 1 can control the in-vehicle equipment provided in vehicle 1. Examples of such in-vehicle equipment include, but are not limited to, in-vehicle air conditioning equipment, in-vehicle audio equipment, and in-vehicle lighting equipment. Controller 40 and electrode device 3 configured as described above may constitute a measuring system provided in steering wheel 2.

The grip detecting circuit may be a grip detector that detects a touch on electrode device 3 by the driver’s hand based on a detection signal output from electrode 32. In this case, electrode 32 may function as a grip detecting electrode as well, and the grip detecting circuit may be able to determine whether the driver’s hand is in contact with electrode device 3 based on a change in the electrostatic capacity between the driver’s hand and the grip detecting electrode. Furthermore, the grip detecting circuit may be able to detect the position where the driver’s hand has touched with the use of a plurality of grip detecting electrodes. The grip detecting circuit can display information indicating that the driver’s hand is not in contact with steering wheel 2 on an in-vehicle monitor provided in vehicle 1.

While electrode device 3 configured as described above can be mounted into or removed from recess 21 of rim 20, when electrode device 3 is placed in recess 21 of rim 20, terminal 37 of circuit board 36 becomes electrically connected to terminal 38a of wires 38, and engaging portion 35 engages with engagement receiving portion 22 of recess 21. With this configuration, electrode device 3 becomes attached to rim 20.

In electrode device 3 according to the example described above, cover 33 is laminated on base 31, but the present embodiment is not limited to this configuration. As shown in FIG. 7, cover 33 may be further laminated with outer cover material 25a. FIG. 7 is a sectional view showing steering wheel 2a that includes electrode device 3a in which outer cover material 25a is laminated.

The outer peripheral surface of steering wheel 2a may be constituted by outer cover material 25, and cover 33 of electrode device 3a may also be laminated with outer cover material 25a. Outer cover materials 25 and 25a are made, for example, of a resin material or leather.

In this case, in order to detect the driver’s biometric information, electrode 32 may be exposed from outer cover material 25a so as to form a portion of the outer peripheral surface of rim 20 of steering wheel 2a. According to the present embodiment, a portion of electrode 32 is exposed from outer cover material 25a. With this configuration, the outer peripheral surface of rim 20 of steering wheel 2a can be made of the same material as the outer peripheral surface of electrode device 3a. This configuration, therefore, makes it less likely that the driver has a sense of discomfort when he or she grips steering wheel 2a.

Outer cover material 25 wrapped around steering wheel 2a may be formed integrally with outer cover material 25a laminated on cover 33 of electrode device 3a, or outer cover material 25 and outer cover material 25a may be separate members. In electrode device 3a, the laminate structure in which outer cover material 25a and cover 33 are combined may be one example of the exterior member. Herein, according to the present embodiment, cover 33 does not need to be provided, and outer cover material 25a alone may be laminated on base 31. In this case, outer cover material 25a may be one example of the exterior member. Furthermore, in this case, outer cover material 25a differs from base 31 only in terms of its material, and outer cover material 25a has the same function as cover 33.

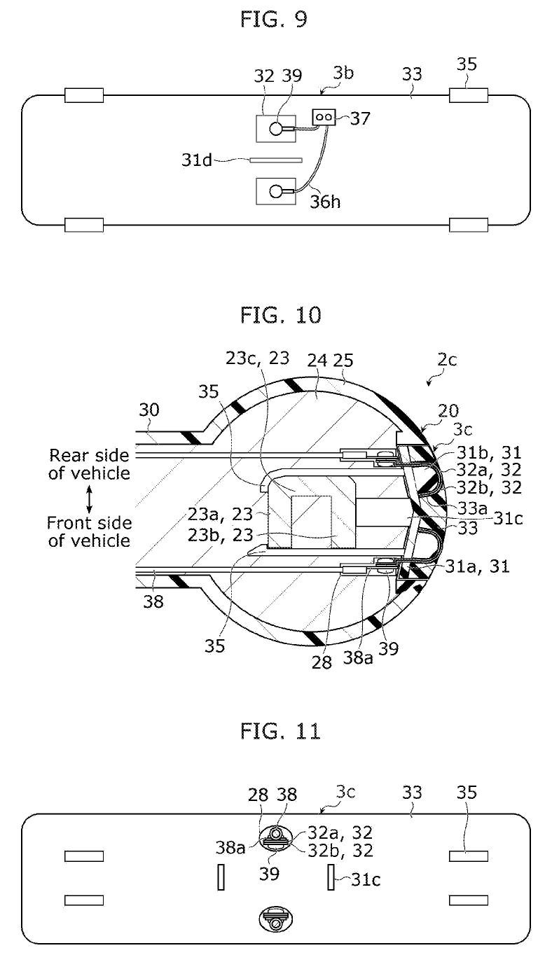

In the example described above, electrode device 3 and so forth include circuit board 36, but the present embodiment is not limited to this configuration. As shown in FIG. 8 and FIG. 9, electrode device 3b does not have to include the circuit board described above. FIG. 8 is a sectional view showing electrode device 3b in which electrode 32 and wire 38 are directly connected to each other. FIG. 9 is a plan view showing the back surface of electrode device 3b shown in FIG. 8.

In this case, electrode 32 may be electrically connected to wire 36h via connector 39. Wire 36h electrically connects electrode 32 and terminal 37.

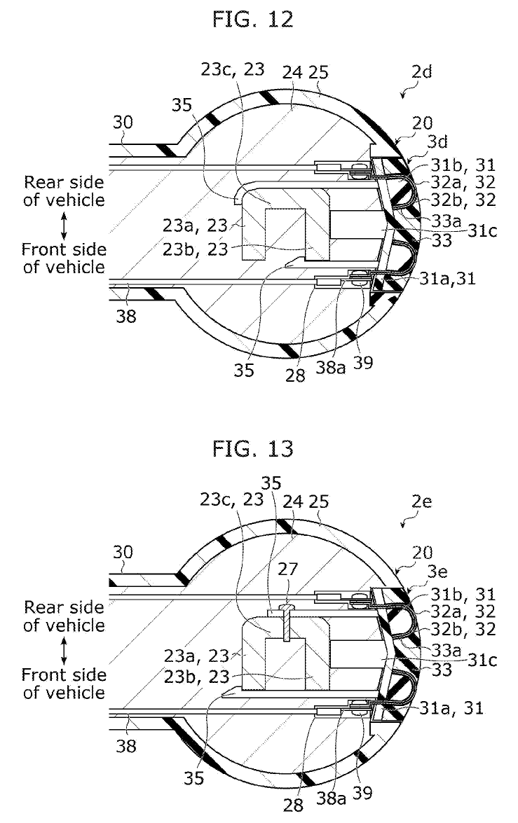

In the example described above, engaging portion 35 engages with recess 21 of steering wheel 2 and so forth, but the present embodiment is not limited to this configuration. For example, as shown in FIG. 10 to FIG. 13, engaging portion 35 may engage with metal core 23 of steering wheel 2c,2d, or 2e. FIG. 10 is a sectional view showing steering wheel 2c that includes electrode device 3c in which engaging portion 35 engages with metal core 23. FIG. 11 is a plan view showing the back surface of electrode device 3c shown in FIG. 10. FIG. 12 is a sectional view showing another steering wheel 2d that includes electrode device 3d in which engaging portion 35 engages with metal core 23. FIG. 13 is a sectional view showing yet another steering wheel 2e that includes electrode device 3e in which engaging portion 35 engages with metal core 23.

In this case, as shown in FIG. 10 and FIG. 11, electrode device 3c may include a pair of engaging portions 35.

Specifically, metal core 23 may include inner portion 23a that is coupled to spoke 30, outer portion 23b that is disposed toward the outer side of inner portion 23a, and coupling portion 23c that couples inner portion 23a and outer portion 23b. Metal core 23 may have a U-shaped section formed by inner portion 23a, outer portion 23b, and coupling portion 23c. The pair of engaging portions 35 may arrayed in the widthwise direction of electrode device 3c so as to sandwich metal core 23. In this case, one engaging portion 35 of the pair of engaging portions 35 may engage with inner portion 23a of metal core 23, other engaging portion 35 of the pair of engaging portions 35 may engage with coupling portion 23c of metal core 23.

Meanwhile, in electrode device 3d shown in FIG. 12, one engaging portion 35 of the pair of engaging portions 35 may engage with outer portion 23b of metal core 23, other engaging portion 35 of the pair of engaging portions 35 may engage with coupling portion 23c of metal core 23.

The engagement between other engaging portion 35 and coupling portion 23c of metal core 23 may be such that other engaging portion 35 is merely hooked on coupling portion 23c.

Meanwhile, in electrode device 3e shown in FIG. 13, the engagement between other engaging portion 35 and coupling portion 23c of metal core 23 may be such that other engaging portion 35 is fixed to coupling portion 23c by tightening member 27, such as a screw.

When configured as described above, electrode devices 3c, 3d, and 3e do not have to include a circuit board. In this case, one wire 38 of the pair of wires 38 is disposed inside steering wheel 2c or 2d on the side that faces toward the front of vehicle 1, and other wire 38 of the pair of wires 38 is disposed inside steering wheel 2c or 2d on the side that faces toward the rear of vehicle 1. Terminal 38a provided at the leading end of one of the pair of wires 38 is electrically connected to one of the pair of electrodes 32 via connector 39. Terminal 38a provided at the leading end of the other of the pair of wires 38 is electrically connected to the other of the pair of electrodes 32 via connector 39. The portion where terminal 38a of wire 38 connected by connector 39 and wire 38 are connected is covered by protective tube 28.

For example, in the example shown in FIG. 10, when steering wheel 2c is to be obtained, first, electrode device 3c is coupled to metal core 23. At this point, one engaging portion 35 of the pair of engaging portions 35 engages with inner portion 23a of metal core 23, other engaging portion 35 of the pair of engaging portions 35 engages with coupling portion 23c of metal core 23. Furthermore, support portion 31c of base body 31a extends toward metal core 23 and abuts metal core 23. This configuration can provide an attachment structure composed of metal core 23 and electrode device 3c attached to metal core 23. Next, foam is formed by urethane on the attachment structure, and thus foam 24 is formed around metal core 23, and recess 21 excluding electrode device 3c is formed in foam 24. Then, outer cover material 25 is wrapped around foam 24, and steering wheel 2c can thus be obtained.

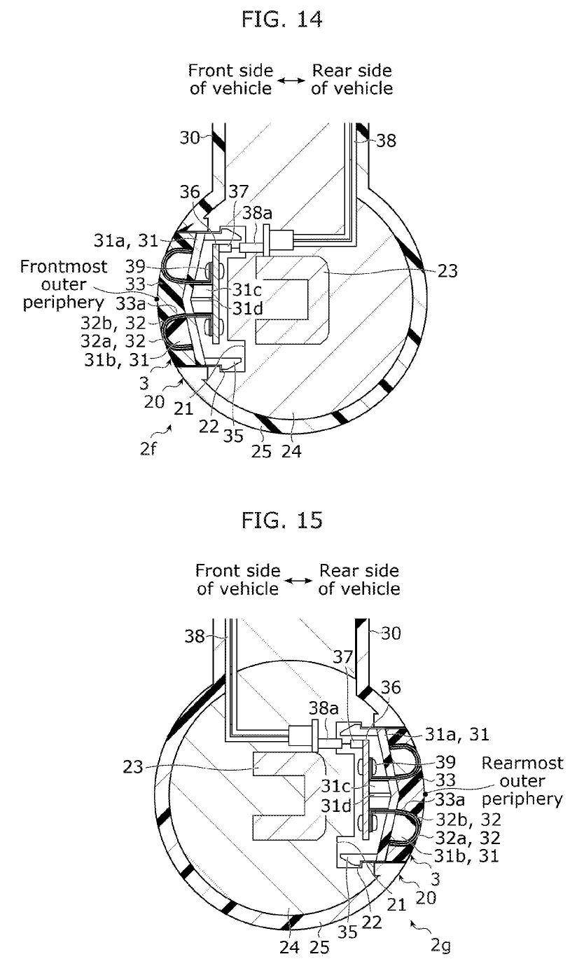

In the example described above, electrode device 3 and so forth are disposed on the outermost periphery of rim 20, but the present embodiment is not limited to this configuration. For example, as shown in FIG. 14, recess 21 may be formed in steering wheel 2f on the side that faces toward the front of vehicle 1. FIG. 14 is a sectional view showing steering wheel 2f that includes electrode device 3 that is disposed on the side that faces toward the front of vehicle 1.

In this case, electrode device 3 mounted in recess 21 is disposed on steering wheel 2f on the side that faces toward the front of vehicle 1. In this case, the pair of electrodes 32 is disposed on steering wheel 2f such that the frontmost outer periphery of steering wheel 2f passes between the pair of electrodes 32.

In the example shown in FIG. 14, electrode device 3 includes circuit board 36, but the present embodiment is not limited to this configuration. For example, electrode device 3 shown in FIG. 14 may be replaced with electrode device 3b shown in FIG. 8 that does not include a circuit board.

Furthermore, as shown in FIG. 10 to FIG. 13, the pair of engaging portions 35 may engage with metal core 23 of steering wheel 2c, 2d, or 2e. In this case, the pair of engaging portions 35 may engage with inner portion 23a and outer portion 23b so as to sandwich metal core 23.

In the example described above, electrode device 3 and so forth are disposed on the outermost periphery of rim 20, but the present embodiment is not limited to this configuration. For example, as shown in FIG. 15, recess 21 may be formed in steering wheel 2g on the side that faces toward the rear of vehicle 1. FIG. 15 is a sectional view showing steering wheel 2g that includes electrode device 3 that is disposed on the side that faces toward the rear of vehicle 1.

In this case, electrode device 3 mounted in recess 21 is disposed on steering wheel 2g on the side that faces toward the rear of vehicle 1. In this case, the pair of electrodes 32 is disposed on steering wheel 2g such that the rearmost outer periphery of steering wheel 2g passes between the pair of electrodes 32.

In the example shown in FIG. 15, electrode device 3 includes circuit board 36, but the present embodiment is not limited to this configuration. For example, electrode device 3 shown in FIG. 15 may be replaced with electrode device 3b shown in FIG. 8 that does not include a circuit board.

Furthermore, as shown in FIG. 10 to FIG. 13, the pair of engaging portions 35 may engage with metal core 23 of steering wheel 2c,2d, or 2e. In this case, the pair of engaging portions 35 may engage with inner portion 23a and outer portion 23b so as to sandwich metal core 23.

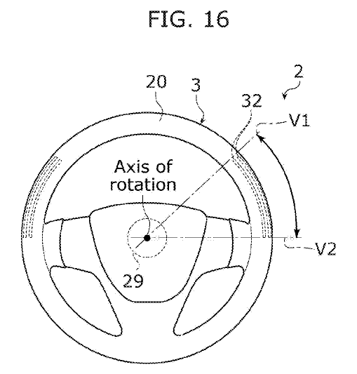

In the example described above with reference to FIG. 1, when rim 20 is viewed as a clock, one of the pair of electrodes 32 is disposed in the area of rim 20 that corresponds to the range of from around 2 o’clock to around 4 o’clock, and the other of the pair of electrodes 32 is disposed in the area of rim 20 that corresponds to the range of from around 8 o’clock to around 10 o’clock, but the present embodiment is not limited to this configuration. For example, the ranges in which the pair of electrodes 32 is disposed may be set as shown in FIG. 16. FIG. 16 is a diagram showing the range of electrode 32 disposed on rim 20 in its neutral position.

In this case, with the axis of rotation of rim 20 (the axis of hub 29) regarded as the center, angle α formed by dashed-dotted straight line V1 connecting one end of electrode 32 and the axis of hub 29 and dashed-dotted straight line V2 connecting the other end of electrode 32 and the axis of hub 29 may be set to be greater than or equal to 30° and smaller than or equal to 90°.

It is to be noted that the content described above according to the present embodiment can be combined as appropriate.

### Workings and Advantageous Effects

Next, the workings and the advantageous effects of electrode devices 3 to 3e and steering wheels 2, 2a, and 2c to 2g according to the present embodiment will be described.

For example, in a case in which the conductive rubber-coated fabric according to PTL 1 is to be mounted on the rim of a steering wheel, in one conceivable configuration, the conductive rubber-coated fabric is sewn to the surface of the steering wheel cover wrapped around the rim with sewing thread. When the driver grips the steering wheel, since the conductive rubber-coated fabric and the sewing thread are on the surface of the steering wheel cover, the design sophistication of the steering wheel may disadvantageously be reduced, or the accuracy of detecting the biometric information may disadvantageously be compromised as the driver’s hand is caught by the sewing thread and becomes unable to touch the conductive rubber-coated fabric.

As described above, electrode devices 3 to 3e according to technique 1 of the present embodiment are electrode devices 3 to 3e to be disposed in steering wheels 2, 2a, and 2c to 2g provided in vehicle 1 with a portion of electrode devices 3 to 3e being exposed from a surface of steering wheels 2, 2a, and 2c to 2g, and electrode devices 3 to 3e include electrode 32, base 31 on which electrode 32 is disposed, and an exterior member disposed on base 31 so as to support electrode 32. Base 31 or the exterior member includes engaging portion 35 that engages with steering wheels 2, 2a, and 2c to 2g, electrode 32 is exposed from the exterior member such that the exterior member forms a portion of the outer peripheral surface of steering wheels 2, 2a, and 2c to 2g when electrode devices 3 to 3e are disposed in steering wheels 2, 2a, and 2c to 2g.

According to this configuration, electrode devices 3 to 3e can be disposed in steering wheels 2, 2a, and 2c to 2g. This configuration can make the surface of steering wheels 2, 2a, and 2c to 2g smooth and thus renders it unnecessary to sew a conductive rubber-coated fabric to the surface of a steering wheel cover with sewing thread as in the related art. Accordingly, the present embodiment can keep the design sophistication of steering wheels 2, 2a, and 2c to 2g from being reduced.

Furthermore, since the surface of steering wheels 2, 2a, and 2c to 2g is smooth, this configuration allows the driver to more easily grip steering wheels 2, 2a, and 2c to 2g and touch electrode 32 with his or her hand. The present embodiment thus makes a situation less likely in which sewing thread keeps the driver’s hand from touching a conductive rubber-coated fabric or the driver’s hand is caught by a conductive rubber-coated fabric and the accuracy of detecting the driver’s biometric information is compromised as a result, as in the related art.

Accordingly, electrode devices 3 to 3e according to the present disclosure can keep the design sophistication of steering wheels 2, 2a, and 2c to 2g from being reduced and keep the accuracy of detecting biometric information from being compromised.

In particular, since the surface of steering wheel 2 can be made smooth, this configuration is less likely to give the driver a sense of discomfort when the driver grips steering wheels 2, 2a, and 2c to 2g, and thus the improvement in how the driver feels when touching steering wheels 2, 2a, and 2c to 2g can be expected.

Meanwhile, electrode devices 3 to 3e according to technique 2 of the present embodiment are electrode devices 3 to 3e according to technique 1. In this case, electrode 32 includes conductive layer 32a formed of a conductive elastomer or a metal plate.

According to this configuration, the driver’s biometric information can be obtained when the driver grips steering wheels 2, 2a, and 2c to 2g.

Meanwhile, electrode devices 3 to 3e according to technique 3 of the present embodiment are electrode devices 3 to 3e according to technique 2. In this case, electrode 32 further includes conductive base fabric 32b, and conductive layer 32a is laminated on conductive base fabric 32b.

According to this configuration, as conductive layer 32a is laminated on conductive base fabric 32b, this configuration makes it possible to reduce the distance between conductive base fabric 32b and the driver’s hand as much as possible.

For example, when conductive layer 32a is formed of a conductive elastomer, using conductive base fabric 32b having a lower electric resistance than a conductive elastomer makes it easier to obtain an electrical signal from the driver’s hand.

Meanwhile, electrode devices 3 to 3e according to technique 4 of the present embodiment are electrode devices 3 to 3e according to any one of techniques 1 to 3. In this case, recess 21 is formed in the surfaces of steering wheels 2, 2a, and 2c to 2g, electrode devices 3 to 3e are disposed in recess 21, and engaging portion 35 engages with engagement receiving portion 22 formed in recess 21.

According to this configuration, as electrode devices 3 to 3e are inserted into recess 21 formed in steering wheels 2, 2a, and 2c to 2g, engaging portion 35 and engagement receiving portion 22 engage with each other, and thus electrode devices 3 to 3e can be attached to steering wheels 2, 2a, and 2c to 2g with ease. Therefore, the present embodiment makes it easier to assemble steering wheels 2, 2a, and 2c to 2g.

Meanwhile, electrode devices 3c to 3e according to technique 5 of the present embodiment are electrode devices 3c to 3e according to any one of techniques 1 to 4. In this case, engaging portion 35 engages with metal core 23 of steering wheels 2c to 2e.

According to this configuration, since engaging portion 35 engages with metal core 23, electrode devices 3c to 3e can be fixed firmly to metal core 23.

Meanwhile, electrode device 3a according to technique 6 of the present embodiment is electrode device 3a according to any one of techniques 1 to 5. In this case, the exterior member includes cover 33 and outer cover material 25a laminated on cover 33, the outer peripheral surface of steering wheel 2a is formed by outer cover material 25a, and electrode 32 is exposed from outer cover material 25a so as to form a portion of the outer peripheral surface.

According to this configuration, since the surface of steering wheel 2a can be made smooth, this configuration is less likely to give the driver a sense of discomfort when the driver grips steering wheel 2a, and thus the improvement in how the driver feels when touching steering wheel 2a can be expected.

Furthermore, the outer peripheral surface of rim 20 of steering wheel 2a can be made of the same material as the outer peripheral surface of electrode device 3a. Therefore, this configuration is less likely to give the driver a sense of discomfort when the driver grips steering wheel 2a, and thus the improvement in how the driver feels when touching steering wheel 2a can be expected

Meanwhile, electrode devices 3 to 3e according to technique 7 of the present embodiment are electrode devices 3 to 3e according to any one of techniques 1 to 6. In this case, base 31 includes electrode supporter 31b for disposing electrode 32 thereon, and base body 31a for disposing the exterior member and electrode supporter 31b thereon. Electrode supporter 31b is a protruding member that protrudes from the front surface of base body 31a on which the exterior member is disposed. Base body 31a has formed therein first engaging hole 31a1 that engages with electrode supporter 31b, and second engaging hole 31a3 that engages with the exterior member. Electrode supporter 31b includes first engaging protrusion 31b1 that engages with first engaging hole 31a1. The exterior member includes second engaging protrusion 33b that engages with second engaging hole 31a3.

According to this configuration, as first engaging protrusion 31b1 engages with first engaging hole 31a1, electrode supporter 31b can be attached to base body 31a with ease. Furthermore, as second engaging protrusion 33b engages with second engaging hole 31a3, the exterior member can be attached to base body 31a with ease. Therefore, the configuration above makes it easier to assemble electrode devices 3 to 3e.

Meanwhile, electrode devices 3 to 3e according to technique 8 of the present embodiment are electrode devices 3 to 3e according to technique 7. In this case, base body 31a has formed therein insertion hole 31a2 into which electrode 32 is inserted, electrode 32 extends from electrode supporter 31b to the back surface of base body 31a via insertion hole 31a2, and electrode 32 is electrically connected to wire 38 disposed inside steering wheels 2, 2a, and 2c to 2g.

According to this configuration, electrode 32 and wire 38 can be electrically connected to each other on the back surface of base body 31a. Therefore, the connecting portion where electrode 32 and wire 38 are electrically connected to each other and wire 38 can be disposed on the back surface of base body 31a. Accordingly, the configuration above makes it possible to keep the design sophistication of steering wheels 2, 2a, and 2c to 2g from being compromised and to make the surface of steering wheels 2, 2a, and 2c to 2g smooth.

Meanwhile, electrode devices 3 to 3e according to technique 9 of the present embodiment are electrode devices 3 to 3e according to any one of techniques 1 to 8. In this case, electrode devices 3 to 3e include a pair of electrodes 32. One of the pair of electrodes 32 is disposed on the side that faces toward the front of vehicle 1, and the other of the pair of electrodes 32 is disposed on the side that faces the rear of vehicle 1. The pair of electrodes 32 is disposed in steering wheels 2, 2a, and 2c to 2e such that the outermost periphery of steering wheels 2, 2a, and 2c to 2e passes between the pair of electrodes 32.

According to this configuration, when the driver grips steering wheels 2, 2a, and 2c to 2e, his or her hand comes into contact with electrode 32, and thus this configuration makes it possible to detect the driver’s biometric information. Accordingly, the accuracy of detecting biometric information can be kept from being compromised.

Meanwhile, electrode devices 3 to 3e according to technique 10 of the present embodiment are electrode devices 3 to 3e according to technique 7 or 8. In this case, electrode 32 is disposed so as to be sandwiched between electrode supporter 31b and the exterior member.

According to this configuration, since electrode 32 is supported by electrode supporter 31b and the exterior member, electrode 32 is less likely to be separated from electrode supporter 31b.

Meanwhile, steering wheels 2, 2a, and 2c to 2g according to technique 11 of the present embodiment include electrode devices 3 to 3e according to any one of techniques 1 to 10, and rim 20 having formed therein recess 21 for disposing electrode devices 3 to 3e therein.

Such steering wheels 2, 2a, and 2c to 2g can provide advantageous effects similar to those provided by the electrode devices described above.

Meanwhile, steering wheels 2, 2a, and 2c to 2g according to technique 12 of the present embodiment are steering wheels 2, 2a, and 2c to 2g according to technique 11. In this case, recess 21 is formed in the outermost periphery of rim 20.

According to this configuration, since electrode devices 3 to 3e can be disposed in the outermost periphery of rim 20, when the driver grips steering wheels 2, 2a, and 2c to 2g, his or her hand comes in contact with electrode 32 with ease, and thus this configuration makes it possible to detect the driver’s biometric information. Accordingly, the accuracy of detecting biometric information can be kept from being compromised.

Meanwhile, steering wheel 2f according to technique 13 of the present embodiment is steering wheel 2f according to technique 11. In this case, recess 21 is formed in rim 20 on the side that faces toward the front of vehicle 1.

According to this configuration, electrode device 3 is disposed so as to be less visible from the driver, and thus the design sophistication perceived when the driver views steering wheel 2f from the front can be kept from being compromised.

Meanwhile, steering wheel 2g according to technique 14 of the present embodiment is steering wheel 2g according to technique 11. In this case, recess 21 is formed in rim 20 on the side that faces toward the rear of vehicle 1.

According to this configuration, electrode device 3 is disposed so as to be easily visible from the driver, and thus the driver can grip steering wheel 2g while paying more attention to making contact with electrode 32 of electrode device 3.

Therefore, the driver’s fingers or palm is more likely to make contact with electrode 32 when he or she grips steering wheel 2g, and thus this configuration makes it further possible to obtain the driver’s biometric information. Accordingly, the accuracy of detecting biometric information can be further kept from being compromised.

### Other Variations

Thus far, electrode devices and so forth according to the present disclosure have been described based on the foregoing embodiment, but this embodiment does not limit the present disclosure. Unless departing from the spirit of the present disclosure, an embodiment obtained by making various modifications that a person skilled in the art can conceive of to the embodiment may also be encompassed by the scope of the present disclosure.

For example, according to the foregoing embodiment, conductive layer 32a and conductive base fabric 32b may be affixed to each other by a conductive adhesive.

Furthermore, according to the foregoing embodiment, electrode 32 may be affixed to base 31 by an adhesive, or electrode 32 may be formed integrally with base 31 through insert molding.

Furthermore, according to the foregoing embodiment, base body 31a and electrode supporter 31b may be formed integrally with each other.

It is to be noted that an embodiment obtained by making various modifications that a person skilled in the art can conceive of to the embodiment and an embodiment achieved by combining, as desired, the constituent elements and the functions of the foregoing embodiment within the scope that does not depart from the spirit of the present disclosure are also encompassed by the present disclosure.

While various embodiments have been described herein above, it is to be appreciated that various changes in form and detail may be made without departing from the spirit and scope of the present disclosure as presently or hereafter claimed.

Further Information about Technical Background to This Application

The disclosure of the following patent application including specification, drawings, and claims is incorporated herein by reference in its entirety: Japanese Patent Application No. 2025-022079 filed on February 14, 2025.

### INDUSTRIAL APPLICABILITY

The present disclosure is useful for a steering wheel provided in a vehicle.

1. An electrode device to be disposed partially exposed from a surface of a steering wheel provided in a vehicle, the electrode device comprising:
an electrode;
a base on which the electrode is disposed; and
an exterior member disposed on the base to support the electrode, wherein
the base or the exterior member includes an engaging portion that engages with the steering wheel,
the exterior member forms a portion of an outer peripheral surface of the steering wheel, when the electrode device is disposed in the steering wheel, and
the electrode is exposed from the exterior member to form a portion of the outer peripheral surface.

2. The electrode device according to claim 1, wherein
the electrode includes a conductive layer formed of a conductive elastomer or a metal plate.

3. The electrode device according to claim 2, wherein
the electrode further includes a conductive base fabric, and
the conductive layer is laminated on the conductive base fabric.

4. The electrode device according to claim 1, wherein
a recess is formed in the surface of the steering wheel,
the electrode device is disposed in the recess, and
the engaging portion engages with an engagement receiving portion formed in the recess.

5. The electrode device according to claim 1, wherein
the engaging portion engages with a metal core of the steering wheel.

6. The electrode device according to claim 1, wherein
the exterior member includes a cover and an outer cover material laminated on the cover,
the outer peripheral surface of the steering wheel is formed by the outer cover material, and
the electrode is exposed from the outer cover material to form a portion of the outer peripheral surface.

7. The electrode device according to claim 1, wherein
the base includes an electrode supporter on which the electrode is disposed, and a base body on which the exterior member and the electrode supporter are disposed,
the electrode supporter is a protruding member that protrudes from a front surface of the base body on which the exterior member is disposed,
the base body has formed therein a first engaging hole that engages with the electrode supporter, and a second engaging hole that engages with the exterior member,
the electrode supporter includes a first engaging protrusion that engages with the first engaging hole, and
the exterior member includes a second engaging protrusion that engages with the second engaging hole.

8. The electrode device according to claim 7, wherein
the base body has formed therein an insertion hole into which the electrode is inserted, and
the electrode:
extends from the electrode supporter to a back surface of the base body via the insertion hole; and
is electrically connected to a wire disposed inside the steering wheel.

9. The electrode device according to claim 1, comprising:
a pair of electrodes, each of which is the electrode, one of the pair of electrodes being disposed to face toward a front of the vehicle, an other of the pair of electrodes being disposed to face toward a rear of the vehicle, wherein
the pair of electrodes is disposed in the steering wheel with an outermost periphery of the steering wheel passing between the pair of electrodes.

10. The electrode device according to claim 7, wherein
the electrode is sandwiched between the electrode supporter and the exterior member.

11. A steering wheel comprising:
the electrode device according to claim 1; and
a rim having formed therein a recess in which the electrode device is disposed.

12. The steering wheel according to claim 11, wherein
the recess is formed in an outermost periphery of the rim.

13. The steering wheel according to claim 11, wherein
the recess is formed in the rim on a side that faces toward a front of the vehicle.

14. The steering wheel according to claim 11, wherein
the recess is formed in the rim on a side that faces toward a rear of the vehicle.
